(19) United States
(12) Patent Application Publication
(10) Pub. No.: US 2003/0109494 A1
(43) Pub. Date: Jun. 12, 2003

(54) Ascorbic acid stability

(76) Inventor: Rolland F. Hebert, Seattle, WA (US)

Correspondence Address: ROLLAND HEBERT, 427 BELLEVUE AVE E. SUITE 301, SEATTLE, WA, 98102, US

(21) Appl. No.: 10/313,646

(22) Filed: Dec. 7, 2002

Related U.S. Application Data
(60) Provisional application No. 60/339,124, filed on Dec. 8, 2001.

(51) Int. Cl. A61K 31/721; A61K 31/375

(52) U.S. Cl. 514/59; 514/474

(57) ABSTRACT

Stable ascorbic acid derivatives, useful as active constituents in food, pharmaceutical as well as cosmeceutical applications, are described.

### BACKGROUND-CROSS-REFERENCES TO RELATED APPLICATION

This application claims the benefit of Provisional Patent Application Serial No. 60/339,124 filed on Dec. 8, 2001.

### FIELD OF THE INVENTION

This patent relates to new stable ascorbic acid derivatives with dextran, the processes for obtaining them and to therapeutic uses of these derivatives. More particularly, the invention relates to derivatives resulting from the reaction between ascorbic acid and dextran, their production process, and pharmaceutical compositions that contain them as active principles.

### BACKGROUND OF THE INVENTION

L-ascorbic acid is one of the most potent compounds acting as an antioxidant in biological systems by scavenging active oxygen species and free radicals. L-ascorbic acid is a well-known water-soluble antioxidant that has a whitening effect and serves as a cofactor of prolinehydroxylase to promote synthesis of collagen (see. Quaglino, D. Jr., et al., J. Biol. Chem, p272-345, 1997). These oxygen radicals have been implicated as causative agents for everything from sunburn to aging. They destroy lipid membranes, break down DNA, and inactivate enzymes. Much work has been done in the last two decades documenting the deleterious behavior of oxygen radicals. Ascorbic acid is biologically significant for many reasons, and has been found to have several different activities in the skin, as pointed out by Englard and Seifter, “The Biochemical Functions of Ascorbic Acid” in Ann. Rev. Nutri. 6:365 (1986). This vitamin has been found to be an antioxidant in blocking the lipid peroxidation of the skin, as demonstrated by Kunert and Tappel, “The Effect of Vitamin C on In-Vivo Lipid Peroxidation in Guinea Pigs as Measured by Pentane and Ethane Production” in Lipids 18:271 (1983). Furthermore, a significant amount of research has been published that describes the effects ascorbic acid has upon scavenging oxygen free radicals under a variety of normal and pathological conditions. L-ascorbic acid is also used in various products requiring a long-term antioxidation effect. L ascorbic acid is used in various pharmaceutical as well as cosmeceutical formulations for prevention of damage to the skin from the sun, for prevention and treatment of aging of the skin and for the amelioration of wrinkling of the skin.

L-Ascorbic acid (or vitamin C) is chemically defined as an alpha-ketolactone. The number 2 and 3 carbons are double-bonded and contain an acid-ionizable hydrogen in water (pK=4.2). Ascorbic acid is also a moderately strong reductant.

However, ascorbic acid is well known to be very unstable to thermal as well as oxidative degradation. There is interest in development of new, more stable ascorbic acid derivatives. Since ascorbic acid is so unstable, much work been done concerning the development of ascorbic acid derivatives with enhanced stability while maintaining the antioxidation activity. Notably, a common way to improve the stability of L-ascorbic acid is to convert a 2- or 3-hydroxyl group of L-ascorbic acid to another substituent (see. U.S. Pat. Nos. 5,143,648; 4,780,549; and 4,177,445). Examples of commercially available derivatives of vitamin C include L-ascorbic acid-6-palmitate, 2,6-dipalmitate, 6-stearate, L-ascorbic acid-3-O-ethyl and magnesium L-ascorbic acid-2-phosphate (see U.S. Pat. No. 5,143,648). Thus, the literature describes ascorbic acid compositions formed by using a very low weight percent ascorbic acid, or a nonaqueous solvent, or by using derivatives of ascorbic acid, usually in a solution buffered to a pH above 4.0. See also U.S. Pat. No. 4,367,157 that discloses stabilizing an aqueous ascorbic acid solution by adding monothioglycerol and maintaining the pH between 4 and 7; U.S. Pat. No. 2,400,171 which discloses stabilizing ascorbic acid by converting it to its calcium or zinc salt. Relatively fat-soluble derivatives of ascorbic acid are L-ascorbic acid-6-palmitate, 2,6-dipalmitate and 6-stearate. Despite the improved chemical stability, these derivatives still have a limitation because of their rapid decomposition in vitro. Thus, there is still a need in the art for more stable ascorbic acid derivatives.

In an attempt to overcome these problems, the inventor has surprisingly discovered that water soluble ascorbic acid derivatives synthesized as a result of the chemical reaction between ascorbic acid and dextran as described following are very stable over a long period of time, are easily synthesized and are economical to produce.

### SUMMARY OF THE INVENTION

Briefly stated, the present invention discloses water soluble ascorbic acid derivatives with dextran. In the context of this invention, ascorbic acid refers to both naturally occurring ascorbic acid as well as synthetic ascorbif acid. Dextran is a polysaccharide produced by bacteria growing on sucrose. Natural dextran usually has a high molecular weight. Lower weight dextrans are produced by depolymerization of natural dextran or by synthesis. All dextrans are chemically comprised of alpha-d-glucophyranosyl units. Therapeutically, dextrans are used as plasma volume expanders and are routinely used in medicine. Any type or form of dextran is contemplated in this invention. Ascorbic acid and dextran are commercially available from Sigma-Aldrich Co, St. Louis, Mo.

The water soluble ascorbic acid derivatives with dextran of the present invention have utility as antioxidant agents, as well as in the prevention and/or treatment of a wide variety of conditions associated with low ascorbic acid levels or excessive free radical formation. Thus, in one embodiment, a water soluble ascorbic acid derivative with dextran of this invention is administered to a warm-blooded animal in need thereof to increase ascorbic acid levels. In another embodiment, a water soluble ascorbic acid derivative with dextran is administered to a warm-blooded animal in need thereof to prevent and/or treat a condition associated with increased free radical formation.

In yet a further embodiment, a water soluble ascorbic acid derivative with dextran is used as a vitamin supplement in mammals and fish.

In another embodiment, a water soluble ascorbic acid derivative with dextran is also used in various products requiring a long-term antioxidation effect.

In still a further embodiment, a synthetic method for the manufacture of water soluble ascorbic acid derivative with dextran is disclosed.

Other aspects of the present invention will become evident upon reference to the detailed description.

### BRIEF DESCRIPTION OF THE DRAWING

FIG. 1 shows stability data over a twelve month period of ascorbic acid derivatives of example 1 and example 2.

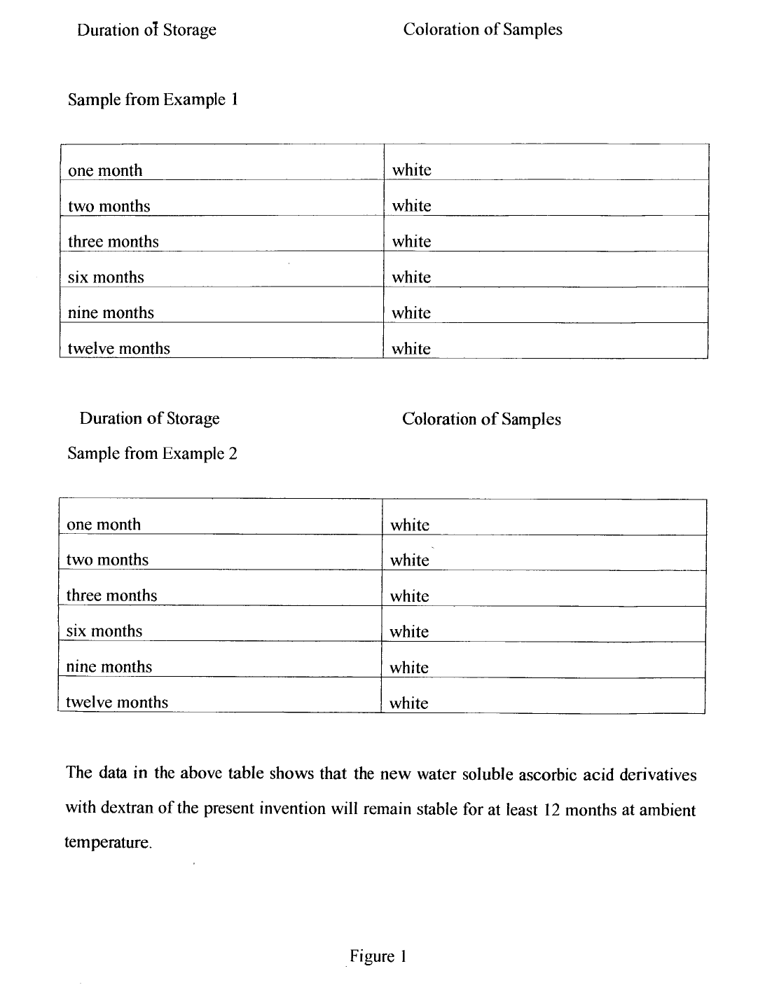

### DETAILED DESCRIPTION OF THE INVENTION

As mentioned above, this invention is generally directed to new, stable water soluble ascorbic acid derivatives with dextran. Such water soluble ascorbic acid derivatives with dextran act as antioxidants, and therefor have utility in the prevention and/or treatment of a wide variety of conditions associated with free radical formation in warm-blooded animals, including humans.

As used herein, the term “conditions” includes diseases, injuries, disorders, indications and/or afflictions that are associated with free radical formation. Conditions “free radical formation” are those conditions that result, either directly or indirectly, in high levels of free radicals. The term “treat” or “treatment” means that the symptoms associated with one or more conditions associated with free radical formation are alleviated or reduced in severity or frequency, and the term “prevent” means that subsequent occurrence of such symptoms are avoided or that the frequency between such occurrences is prolonged.

In one embodiment of this invention, synthetic routes for the manufacture of stable water soluble ascorbic acid derivatives with dextran are disclosed. In this regard, highly pure stable water soluble ascorbic acid derivatives with dextran can be manufactured in high yield by mixing ascorbic acid with dextran. Generally this synthesis may be achieved by dissolving dextran (average molecular weight 15,000) in water at pH 5.2 and 21 degrees C. to which ascorbic acid is added with constant stirring at 21 degrees C. and pH 2.46. The solution is dried by freeze drying and results in a stable powder.

In another manner of this present embodiment, dextran (average molecular weight 15,000) (and equal in weight to the amount of ascorbic acid that will be added later) is dissolved in water at pH 5.2 and 21 degrees C. Ascorbic acid (equal in weight to the dextran) is added to this solution with constant stirring for 30 minutes at 21 degrees C. and pH 2.46. The solution is dried by freeze drying and results in a stable powder.

Another method for the synthesis of stabilized ascorbic acid consists of dissolving dextran (average molecular weight 15,000) (one half the amount of the weight of ascorbic acid to be added for stabilization) in water at pH 5.2 and 21 degrees C. Ascorbic acid (twice the amount of dextran) is added to this solution with constant stirring for 30 minutes at 21 degrees C. and pH 2.62. The solution is dried by freeze drying and results in a stable powder.

In a more preferable embodiment, the synthesis is carried out in the following manner that lends itself easily to scale up on an industrial level. Dextran (2 grams, average molecular weight 15,000) is dissolved in water (100 ml) at pH 5.2 and 21 degrees C. Ascorbic acid (2 grams) is added to this solution with constant stirring for 30 minutes at 21 degrees C. and pH 2.46. The solution is dried by freeze drying and results in a stable powder.

In an even more preferable embodiment, the synthesis is carried out in the following manner that also lends itself easily to scale up on an industrial level. Dextran (1 gram, average molecular weight 15,000) is dissolved in water (100 ml) at pH 5.2 and 21 degrees C. Ascorbic acid (2 grams) is added to this solution with constant stirring for 30 minutes at 21 degrees C. and pH 2.62. The solution is dried by freeze drying and results in a stable powder. (Drying may of course also be carried out by other methods than freeze drying and that are all well known in the art.)

The amount of dextran that may be used to stabilize ascorbic acid in the synthetic process of this present invention can range from 1% of the weight of ascorbic acid to be stabilized to 100% of the weight of the ascorbic acid. For reasons of economy, the least amount of dextran needed to stabilize the ascorbic acid is the preferred quantity. The pH of the dextran solutions in which the ascorbic acid is dissolved may range from pH 1 to pH 6.9 but more preferably from pH2.3-pH3.5.

The water soluble ascorbic acid derivatives with dextran of this invention may be used to prevent and/or treat a variety of conditions associated with excessive free radical formation.

Accordingly, the water soluble ascorbic acid derivatives with dextran of this invention are believed effective in preventing and/or treating the above conditions due to their ability to act as antioxidants. To this end, the water soluble ascorbic acid derivatives with dextran of the present invention may be used for pharmaceutical, prophylactic and/or cosmetic purposes, and are administered to a warm-blooded animal in an effective amount to achieve a desired result. In the case of pharmaceutical administration, an effective amount is a quantity sufficient to treat the symptoms of a condition and/or the underlying condition itself. An effective amount in the context of prophylactic administration means an amount sufficient to avoid or delay the onset of a condition and/or its symptoms. Lastly, an effective amount with regard to cosmetic administration is an amount sufficient to achieve the desired cosmetic result.

In a preferred embodiment, the water soluble ascorbic acid derivatives with dextran of the present invention are administered to a warm-blooded animal as a pharmaceutical, prophylactic or cosmetic composition. Administration may be accomplished by systemic or topical application, with the preferred mode dependent upon the type and location of the conditions to be treated. Frequency of administration may vary, and is typically accomplished by daily administration.

Systemic administration may be achieved, for example, by injection (e.g., intramuscular, intravenous, subcutaneous or intradermal) or oral delivery of the composition to the warm-blooded animal. Suitable carriers and diluents for injection are known to those skilled in the art, and generally are in the form of an aqueous solution containing appropriate buffers and preservatives. Oral delivery is generally accomplished by formulating the composition in a liquid or solid form, such as a tablet or capsule, by known formulation techniques. Daily dosages of compositions of the present invention may vary depending on the condition of the patient, the patient's health history and other medications, and the like. In general, dosages of compositions of the present invention are administered to mammals in need thereof at dosage levels of approximately 5 mg to 20 grams per day, and more preferably at dosage levels of approximately 100 mg to 3 grams per day. Treatment protocols may involve a single daily dosage, or may involve equally divided doses throughout the day.

Topical administration may be accomplished, for example, by formulating the composition as solution, cream, gel, ointment, powder, paste, gum or lozenge using techniques known to those skilled in the formulation field. As used herein, topical administration includes delivery of the composition to mucosal tissue of the mouth, nose and throat by, for example, spray or mist application, as well as to the vagina and rectum by, for example, suppository application.

In addition, the new stable ascorbic acid derivatives with dextran of this present invention can also be used to make stable solutions of ascorbic acid that can be used to prevent oxidation reactions in foodstuffs. For example, meat, poultry, fish, can be dipped in a solution of stable ascorbic acid derivatives with dextran thus coating the product with a layer of ascorbic acid providing antioxidant protection against discoloration of the product. The stable ascorbic acid derivatives with dextran of this present invention can also be used as an additive to foodstuffs, beverages, feeds for animals and fish and the like.

It has now surprisingly and unexpectedly been found that stable ascorbic acid derivatives with dextran have good characteristics that are such as to render them particularly suitable both for use in pharmaceutical formulations and for preparative applications. Owing to their simple conception and low costs, the procedures described in this invention easily lend themselves to working out methods of preparation on an industrial scale.

The examples given herein below illustrate the preparation of two stable ascorbic acid derivatives with dextran. Only a few of the many possible embodiments that may be anticipated are shown by these examples that are intended to define, in a non-limiting sense, the scope encompassed by the invention.

These examples are given to illustrate the present invention, but not by way of limitation. Accordingly, the scope of this invention should be determined not by the embodiments illustrated, but rather by the appended claims and their legal equivalents.

### EXAMPLE 1

Dextran (2 grams, average molecular weight 15,000) was dissolved in water (100 ml) at pH 5.2 and 21 degrees C. Ascorbic acid (2 grams) was added to this solution with constant stirring for 30 minutes at 21 degrees C. and pH 2.46. The solution was dried by freeze drying and resulted in a stable powder. The resulting powder was stored at room temperature in clear glass vials without special precautions to exclude air or oxygen from contacting the powder (i.e., without blanketing the resulting powder with nitrogen to reduce the potential of oxidation by air or oxygen). Ascorbic acid stability was determined by the absence of an off-white to brown coloration characteristic of degraded ascorbic acid.

### EXAMPLE 2

Dextran (1 gram, average molecular weight 15,000) was dissolved in water (100 ml) at pH 5.2 and 21 degrees C. Ascorbic acid (2 grams) was added to this solution with constant stirring for 30 minutes at 21 degrees C. and pH 2.62. The solution was dried by freeze drying and resulted in a stable powder. The resulting powder was stored at room temperature in clear glass vials without special precautions to exclude air or oxygen from contacting the powder (i.e., without blanketing the resulting powder with nitrogen to reduce the potential of oxidation by air or oxygen). Ascorbic acid stability was determined by the absence of an off-white to brown coloration characteristic of degraded ascorbic acid.

I claim:

1. A composition comprising ascorbic acid with dextran.

2. The composition of claim 1 wherein the amount of dextran used to stabilize the ascorbic acid is between.01% to 100% of the weight of ascorbic acid.

3. The composition of claim 1 wherein the amount of dextran used to stabilize the ascorbic acid is between.10% to 80% of the weight of ascorbic acid.

4. The composition of claim 1 wherein the amount of dextran used to stabilize the ascorbic acid is between.30% to 60% of the weight of ascorbic acid.

5. The composition of claim 1 wherein the amount of dextran used to stabilize the ascorbic acid is between.40% to 50% of the weight of ascorbic acid.

6. A pharmaceutical composition for the treatment or prophylaxis of a condition in warm-blooded animals that responds to inhibition of oxygen free radicals, that comprises an amount of a composition according to claim 1 or of a pharmaceutically acceptable salt thereof according to claim 1 that is active against conditions that respond to inhibition of oxygen free radicals, together with a pharmaceutically acceptable carrier.

7. A method for the treatment of a condition which is treatable by inhibition of oxygen free radicals, which comprises administration of a prophylactically or therapeutically active oxygen free radical-inhibiting amount of a composition of claim 1 or of a pharmaceutically acceptable salt thereof according to claim 1 to a warm-blooded animal requiring such treatment.

8. A method of improving skin appearance, comprising applying an effective amount of the composition of claim 1 to the skin of a warm-blooded animal requiring such treatment.

9. A method of preventing sun damage to skin, comprising applying an effective amount of the composition of claim 1 to the skin of to a warm-blooded animal requiring such treatment.

10. A method of treating sun damage to skin, comprising applying an effective amount of the composition of claim 1 to the skin of to a warm-blooded animal requiring such treatment.

11. A method of supplementing ascorbic acid to the diet, comprising adding an effective amount of the composition of claim 1 to the food, beverages and animal and fish feeds.

12. A method for the synthesis of the composition of claim 1 that comprises dissolving an amount of dextran (equal to the amount of ascorbic acid to be stabilized) in water at pH 5.2 and 21 degrees C. and adding ascorbic acid to this solution with constant stirring for 30 minutes at 21 degrees C. and pH 2.46 and further comprises freeze drying or spray drying the solution to obtain a stable powder.

13. A method for the synthesis of the composition of claim 1 that comprises dissolving an amount of dextran (equal to one half that of ascorbic acid to be stabilized) in water at pH 5.2 and 21 degrees C. and adding ascorbic acid to this solution with constant stirring for 30 minutes at 21 degrees C. and pH 2.62 and further comprises freeze drying or spray drying the solution to obtain a stable powder.
